SEQUENCE LISTING (9 sequences)

SEQ ID NO: 1 - DNA, 19 bp, synthetic construct
  misc_feature 1..19: note = NON-MOE
  source 1..19: mol_type = other DNA
gtaggtaaaaacctaatat

SEQ ID NO: 2 - DNA, 20 bp, synthetic construct
  misc_feature 1..20: note = NON-MOE
  source 1..20: mol_type = other DNA
gttcgtttagagaacagatc

SEQ ID NO: 3 - DNA, 21 bp, synthetic construct
  misc_feature 1..21: note = NON-MOE
  source 1..21: mol_type = other DNA
taaagttcgtttagagaacag

SEQ ID NO: 4 - DNA, 15 bp, synthetic construct
  misc_feature 1..15: note = NON-MOE
  source 1..15: mol_type = other DNA
agccctgtatacgac

SEQ ID NO: 5 - DNA, 19 bp, synthetic construct
  misc_feature 1..19: note = MOE
  source 1..19: mol_type = other DNA
gtaggtaaaaacctaatat

SEQ ID NO: 6 - DNA, 20 bp, synthetic construct
  misc_feature 1..20: note = NON-MOE
  source 1..20: mol_type = other DNA
cgtttagagaacagatctac

SEQ ID NO: 7 - DNA, 20 bp, synthetic construct
  misc_feature 1..20: note = MOE
  source 1..20: mol_type = other DNA
cattgtagatgtcaaaagcc

SEQ ID NO: 8 - DNA, 21 bp, synthetic construct
  misc_feature 1..21: note = NON-MOE
  source 1..21: mol_type = other DNA
ctccctcatggtggcagttga

SEQ ID NO: 9 - DNA, 18 bp, synthetic construct
  misc_feature 1..18: note = NON-MOE
  source 1..18: mol_type = other DNA
cggcatgtctattttgta

(19) United States
(12) Patent Application Publication
(10) Pub. No.: US 2024/0093198 A1
(43) Pub. Date: Mar. 21, 2024

(54) TGF-BETA THERAPEUTICS FOR AGE DISEASE

(71) Applicant: GMP BIOTECHNOLOGY LIMITED, Hong Kong (CN)

(72) Inventor: Vuong TRIEU, Agoura Hills, CA (US)

(21) Appl. No.: 18/518,560

(22) Filed: Nov. 23, 2023

Related U.S. Application Data
(63) Continuation of application No. PCT/US2022/030512, filed on May 23, 2022.
(60) Provisional application No. 63/282,634, filed on Nov. 23, 2021.
(60) Provisional application No. 63/192,175, filed on May 24, 2021.

(51) Int. Cl. C12N 15/113; A61K 31/366; A61K 45/06

(52) U.S. Cl. CPC C12N 15/1136; A61K 31/366; A61K 45/06; C12N 2310/11; C12N 2320/35

(57) ABSTRACT

This invention relates to methods, compositions, kits and uses of medicaments for treating or ameliorating the symptoms of age disease in a human or animal, or for treating or ameliorating the symptoms of a viral disease, or for inhibiting or suppressing entry or replication of a virus in a cell, or for suppressing an inflammatory response or a cytokine storm. These purposes can be achieved with formulations of agents for inhibiting or suppressing expression of TGF-β. More particularly, this invention discloses compositions, methods and uses for anti-TGF-β agents, such as antisense oligonucleotides against TGF-β, artemisinin, or a combination thereof, in a regimen for age disease.

### SEQUENCE LISTING

This application includes a sequence listing submitted electronically as an xml-ASCII file created on Nov. 23, 2023, named 018988-001US1_SL.xml, which is 12,126 bytes in size and is hereby incorporated by reference in entirety.

### TECHNICAL FIELD

This invention relates to therapeutics for treating or ameliorating symptoms of disease that occurs due to age following an earlier condition. More particularly, this invention discloses compositions and agents for inhibiting or suppressing TGF-beta, which provide improved clinical outcomes for such diseases. This invention provides stable formulations of anti-TGF-beta agents including antisense oligonucleotide compositions and other agents and methods of use for symptoms of disease that occurs due to age following an earlier condition.

### BACKGROUND

Conventional drug methods generally do not sufficiently improve symptoms for long-lasting disease such as caused by COVID type variants and other diseases. In some cases, survivors of an earlier condition have increased risk for age disease, leading to poor survival rates. Further, earlier conditions may affect multiple organs and require several modalities of treatment for later age disease.

Pathology of earlier diseases can be complex and unpredictable. For example, the SAR-CoV-2 infection upregulates TGF-β and Furin. TGF-β locks the cell cycle allowing the virus to replicate. Further, TGF-β drives the expression of Furin-a protease that is required for the cellular entry of SARS-CoV-2. In primary human bronchial epithelial cells, TGF-β1 and TGF-β2 induce expression of Furin. This pathway constitutes a positive loop that keeps spinning off TGF-β resulting in a TGF-β surge.

The TGF-β surge drives the pathology of COVID-19 in a dangerous, out-of-control positive feedback loop. Consequently, TGF-β rises from barely detectable levels before infection to 50,000-100,000 pg/ml during infection. TGF-β drives progression to a pulmonary infection phase, and finally a hyperinflammatory phase. Thus, TGF-β induces scarring in multiple organs including lung, liver, kidney, and brain, which is responsible for long term, late stage post-COVID symptoms.

Because of the unpredictability of the pathology of long-lasting disease, new therapies will require clinical studies for distinct patient populations. Ideally, the most reliable tests are required, such as a double-blind, randomized, placebo-controlled study of patients with age disease. Moreover, such studies generally require highly stable formulations for accurate results.

What is needed are methods and compositions for inhibiting or suppressing factors in the unpredictable pathology of long lasting or age disease. For example, there is a need for methods and compositions for inhibiting the activity of TGF-β and/or suppressing TGF-β related pathologies, which can improve the efficacy for treating or ameliorating the symptoms of long lasting COVID-like and other diseases which follow with age.

There is an urgent need for methods and compositions for inhibiting and/or suppressing TGF-β which provide positive clinical results for treating long lasting disease, as well as other diseases that occur with age. Stable formulations of agents for suppressing TGF-β are needed to reduce damage to various organs, relieve disease action, and reduce long term post-infection symptoms.

### BRIEF SUMMARY

This invention provides therapies for treating or ameliorating symptoms of age disease.

As used herein, age disease refers to diseases or conditions which follow later in time after an earlier disease or condition, or during or after a disease having long lasting symptoms or effects, or diseases or conditions which follow after long lasting symptoms of an earlier condition have ceased. Later in time can be relatively short, such as days, or relatively long, such as years or decades.

In some embodiments, this invention includes agents and compositions for inhibiting or suppressing TGF-beta to provide improved clinical outcomes for age disease.

In further embodiments, this invention provides stable formulations of anti-TGF-beta agents for various therapies for age disease. Examples of anti-TGF-beta agents include TGF-β inhibitors such as antisense oligonucleotides, artemisinin, pharmaceutically acceptable salts forms, esters, polymorphs or stereoisomers thereof, as well as combinations thereof.

In general, the pathology of severe and/or long-lasting disease is unpredictable, therefore new therapies will require clinical studies for distinct patient populations. This disclosure provides results for a highly reliable, double-blind, randomized, placebo-controlled study of patients with long lasting age or respiratory disease.

In further aspects, this disclosure provides highly stable formulations of anti-TGF-beta agents for therapies for age disease. The stable formulations of this invention provide surprisingly improved clinical results. Stable formulations of agents for suppressing TGF-β can be used to reduce damage to various organs, relieve disease action, and reduce long term post-infection symptoms for age disease.

Methods and compositions of this invention can be used for inhibiting or suppressing factors in the unpredictable pathology of age-related disease. In certain embodiments, this disclosure provides methods and compositions for inhibiting the activity of TGF-β and/or suppressing TGF-β related pathologies, which can improve the efficacy for treating or ameliorating the symptoms of COVID-like and other diseases.

Compositions and formulations of this disclosure can be used for inhibiting and/or suppressing TGF-β to provide positive clinical results for treating age disease, as well as other diseases.

Embodiments of this invention include the following:

A process for treating or ameliorating the symptoms of age disease in a human subject or animal in need, the process comprising:
preparing a pharmaceutical composition comprising an agent for inhibiting or suppressing expression of TGF-β; and
administering a therapeutically sufficient amount of the composition to the subject. Use of a composition comprising an agent for inhibiting or suppressing expression of TGF-β for treating or ameliorating the symptoms of an age disease in a human subject or animal. Use of a composition comprising an agent for inhibiting or suppressing expression of TGF-β in the preparation of a medicament for treating or ameliorating the symptoms of an age disease in a human subject or animal. The age disease may be due to a viral disease.

The process or use above, wherein the age disease is due to SARS, MERS, Coronavirus, HIV, Ebola, Cytomegalovirus, human herpes virus type 6, herpes simplex virus HSV-1, herpes simplex virus HSV-2, Epstein-Barr virus, Hepatitis B virus, Enterovirus D68, or Influenza A.

The process or use above, wherein the age disease is long term coronavirus disease, hyperinflammatory immune disease, severe respiratory disease due to viral infection, age-related fibrotic disease, or age-related cancer.

The process or use above, wherein the subject is hospitalized according to one of the following:
WHO COVID-19 Clinical Improvement Ordinal Scale Criteria 3, wherein the subject is hospitalized without oxygen therapy;
WHO COVID-19 Clinical Improvement Ordinal Scale Criteria 4, wherein the subject is hospitalized with oxygen by mask or nasal prongs;
WHO COVID-19 Clinical Improvement Ordinal Scale Criteria 5, wherein the subject is hospitalized with non-invasive mechanical ventilation or high-flow oxygen; and
WHO COVID-19 Clinical Improvement Ordinal Scale Criteria 6, wherein the subject is hospitalized with intubation and mechanical ventilation.

The process or use above, wherein the subject has age greater than 60 years and is hospitalized and presenting at least one medical risk factor selected from:
absolute lymphocyte count ≤1000 cells/mm³;
hypertension;
diabetes;
cardiac failure; and
COPD.

The process or use above, wherein the subject has age greater than 35 years and is hospitalized and exhibiting low PaO₂ less than 77 mmHg.

The process or use above, wherein the disease is multiorgan fibrosis due to aging including any one of lung failure, cardiac failure, kidney failure, and brain cognitive dysfunction.

The process or use above, wherein the subject has long term COVID disease symptoms due to any COVID variant.

The process or use above, wherein the administration or use of the composition is combined with a standard of care treatment for the disease.

The process or use above, comprising any one or more additional medicaments comprising anti-inflammatories, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

The process or use above, wherein the subject upon administration or use has an improved clinical score based on an eight point WHO COVID-19 Clinical Improvement Ordinal Scale at Day 14.

The process or use above, wherein the subject upon the administration or use has an improved inflammatory biomarker.

The process or use above, wherein the administration or use of the composition decreases mortality rate at Day 7, or Day 14, or Day 28.

The process or use above, wherein the administration or use of the composition improves viral load knockdown at Day 7.

The process or use above, wherein the administration or use of the composition increases survival rate at Day 14, or Day 28.

The process or use above, wherein the agent is an antisense oligonucleotide or inhibitor specific for TGF-β1, TGF-β2, or TGF-β3.

The process or use above, wherein the agent for inhibiting or suppressing expression of TGF-β is selected from TGF-β2-specific antisense oligonucleotides SEQ ID NOs:1-9 and chemically-modified variants thereof, artemisinin extract, a pharmaceutically-acceptable salt, salt polymorph, ester, or isomer thereof, an artemisinin formulation, and any combination thereof.

The process or use above, wherein the agent for inhibiting or suppressing expression of TGF-β is an artemisinin formulation, comprising 90-95% pure artemisinin extract, or a pharmaceutically-acceptable salt, salt polymorph, ester, or isomer thereof, and one or more pharmaceutically acceptable excipients.

The process or use above, wherein the excipients comprise any one or more pharmaceutically acceptable excipients selected from diluents, stabilizers, disintegrants and anticaking agents.

The process or use above, wherein the excipients comprise any one or more of microcrystalline cellulose, polysorbate 80, crospovidone, croscarmellose sodium, and magnesium stearate.

The process or use above, wherein the agent for inhibiting or suppressing expression of TGF-β is an artemisinin compound or derivative thereof, or a pharmaceutically-acceptable salt, salt polymorph, ester, or isomer thereof.

The process or use above, wherein the agent for inhibiting or suppressing expression of TGF-β is a compound, or ligand comprising a small molecule or polypeptide, that interacts with Site I of TGF-β comprising Trp30 and/or Site II of TGF-β comprising Arg15, Gln19, and Phe8, or a pharmaceutically-acceptable salt, salt polymorph, ester, or isomer thereof.

The process or use above, wherein the agent for inhibiting or suppressing expression of TGF-β is a polypeptide or peptide mimetic of Site I of TGF-β comprising residues Phe24-Lys37 and/or Site II of TGF-β comprising residues Cys7-G1n19, or a pharmaceutically-acceptable salt, salt polymorph, ester, or isomer thereof.

The process or use above, wherein the agent for inhibiting or suppressing expression of TGF-β is an antibody or antibody fragment with affinity for Site I of TGF-β comprising residues Phe24-Lys37 and/or Site II of TGF-β comprising residues Cys7-G1n19.

The process or use above, wherein the agent for inhibiting or suppressing expression of TGF-β is a compound comprising a sesquiterprene lactone or derivative thereof, or a pharmaceutically-acceptable salt, salt polymorph, ester, or isomer thereof.

The process or use above, wherein the agent for inhibiting or suppressing expression of TGF-β is a compound comprising three isoprenyl groups and one lactone ring, or derivative thereof, or a pharmaceutically-acceptable salt, salt polymorph, ester, or isomer thereof.

The process or use above, wherein the composition is prepared from a lyophilized powder of the agent.

The process or use above, wherein the agent is a TGF-β2-specific antisense oligonucleotide selected from SEQ ID NOs:1-9 and chemically-modified variants thereof, and administered or used by continuous intravenous infusion at a dose of 140 mg/m 2 on Days 1 to 7.

The process or use above, wherein the agent is a TGF-β2-specific antisense oligonucleotide selected from SEQ ID NOs:1-9 and chemically-modified variants thereof, and administered or used by continuous intravenous infusion with a Cmax value of 2-3 μg/mL.

The process or use above, wherein the agent is a TGF-β2-specific antisense oligonucleotide selected from SEQ ID NOs:1-9 and chemically-modified variants thereof, and administered or used by continuous intravenous infusion at a dose of 140 mg/m 2 on Days 1 to 7, either singly or in combination with artemisinin in any form at a dose of 500 mg per day on Days 1 to 5.

The process or use above, comprising suppressing symptoms due to TGF-β induced proteins upon administration or use of the composition.

The process or use above, comprising suppressing symptoms due to any one of long term coronavirus disease, hyperinflammatory immune disease, severe respiratory disease due to viral infection, age-related fibrotic disease, or age-related cancer upon administration or use of the composition.

The process or use above, comprising suppressing symptoms due to multiorgan inflammatory syndrome, cytokine storm, vasculitis, or Kawasaki syndrome upon administration or use of the composition.

The process or use above, comprising suppressing symptoms due to cytokine storm upon administration or use of the composition.

The process or use above, comprising reducing intensive care unit duration upon administration or use of the composition. The intensive care unit duration may be reduced by at least 1 day, or by 1-10 days.

The process or use above, comprising reducing hospitalization duration upon administration or use of the composition. The hospitalization duration may be reduced by at least 1 day, or by 1-10 days.

The process or use above, comprising increasing ventilator-free days upon administration or use of the composition. The ventilator-free days may be increased by at least 1 day, or by 1-10 days.

A pharmaceutical composition for inhibiting or suppressing expression of TGF-β, or for inhibiting or suppressing entry or replication of a virus in a cell, or for inhibiting or suppressing an inflammatory response or cytokine storm, or for treating or ameliorating the symptoms of an age-related disease in a human subject or animal, the composition comprising:
a TGF-β inhibitor, artemisinin, pharmaceutically acceptable salts forms, esters, polymorphs or stereoisomers thereof, and any combination thereof; and
a carrier.

A pharmaceutical composition for inhibiting or suppressing expression of TGF-β in vitro, comprising:
a TGF-β inhibitor, artemisinin, pharmaceutically acceptable salts forms, esters, polymorphs or stereoisomers thereof, and any combination thereof; and
a carrier.

A pharmaceutical composition for inhibiting or suppressing expression of TGF-β in vitro, or for inhibiting or suppressing entry or replication of a virus in a cell in vitro, the composition comprising:
a TGF-β inhibitor, artemisinin, pharmaceutically acceptable salts forms, esters, polymorphs or stereoisomers thereof, and any combination thereof; and
a carrier.

A pharmaceutical composition comprising:
a TGF-β inhibitor, artemisinin, pharmaceutically acceptable salts forms, esters, polymorphs or stereoisomers thereof, and any combination thereof; and
a carrier.

A method for preparing a medicament for inhibiting or suppressing expression of TGF-β, or for inhibiting or suppressing entry or replication of a virus in a cell, or for inhibiting or suppressing an inflammatory response or cytokine storm, or for treating or ameliorating the symptoms of an age-related disease in a human subject or animal, the method comprising:
preparing a TGF-β inhibitor, artemisinin, pharmaceutically acceptable salts forms, esters, polymorphs or stereoisomers thereof, and any combination thereof; and
combining with a carrier.

A method for preparing a medicament for inhibiting or suppressing expression of TGF-β, the method comprising:
preparing a TGF-β inhibitor, artemisinin, pharmaceutically acceptable salts forms, esters, polymorphs or stereoisomers thereof, and any combination thereof; and
combining with a carrier.

The composition above, wherein the TGF-β inhibitor is selected from TGF-β2-specific antisense oligonucleotides SEQ ID NOs:1-9 and chemically-modified variants thereof.

The composition above, wherein the carrier is sterile water for injection, saline, isotonic saline, or a combination thereof.

The composition above, wherein the composition is substantially free of excipients.

The composition above, wherein the composition is stable for at least 14 days in carrier at 37° C.

The composition above, wherein the composition is combined with a standard of care medicament for the disease.

The composition above, comprising any one or more additional medicaments comprising anti-inflammatories, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

The composition above, wherein the age disease is due to a viral disease.

The composition above, wherein the age disease is due to SARS, MERS, Coronavirus, HIV, Ebola, Cytomegalovirus, human herpes virus type 6, herpes simplex virus HSV-1, herpes simplex virus HSV-2, Epstein-Barr virus, Hepatitis B virus, Enterovirus D68, or Influenza A.

The composition above, wherein the age disease is long term coronavirus disease, hyperinflammatory immune disease, severe respiratory disease due to viral infection, age-related fibrotic disease, or age-related cancer.

The composition above, wherein the disease is multiorgan fibrosis due to aging including lung failure, cardiac failure, kidney failure, or brain cognitive dysfunction.

A kit comprising a lyophilized powder in a vial at a content of 250 mg each of one or more TGF-β2-specific antisense oligonucleotides selected from SEQ ID NOs:1-9 and chemically-modified variants thereof.

A kit comprising a lyophilized powder in a vial at a content of 500 mg of artemisinin or a derivative thereof, or a compound, or ligand comprising a small molecule or polypeptide, that interacts with Site II of TGF-β comprising Arg15, Gln19, and Phe8, a sesquiterprene lactone or derivative thereof, or a compound comprising three isoprenyl groups and one lactone ring and derivatives thereof, or a pharmaceutically-acceptable salt, salt polymorph, ester, or isomer thereof, or any combination of any of the foregoing.

### BRIEF DESCRIPTION OF THE DRAWINGS

FIG. 1 shows analysis of new compositions which have been discovered for inhibiting TGF-β using bioinformatic structure-based ligand design to identify and measure primary and alternative binding sites of TGF-β1. The results determined two sites for binding activity: Site 1 included residues Phe24-Lys37, and Site 2 included residues Cys7-G1n19.

FIG. 2 shows a design protocol for a double-blind, randomized, placebo-controlled, multi-center study of antisense oligonucleotide OT-101 in hospitalized COVID-19 patients.

FIG. 3 shows improved rate of survival was achieved in a study of antisense oligonucleotide OT-101 in hospitalized COVID-19 patients.

FIG. 4 shows results for a clinical trial using Artemisinin against COVID. Artemisinin restored long term lung functions due to viral infection in patients with confirmed SARS-CoV-2 infection. These results showed that Artemisinin was effective against COVID.

### DETAILED DESCRIPTION OF THE INVENTION

This invention provides therapies for treating or ameliorating symptoms of age disease. As used herein, age disease refers to diseases which follow after an earlier condition, or a disease with long lasting symptoms, or which follow after long lasting symptoms of an earlier condition.

In some embodiments, this invention includes agents and compositions thereof for inhibiting or suppressing TGF-beta to provide improved clinical outcomes for severe and/or long lasting age or respiratory disease.

In further embodiments, this invention provides stable formulations of anti-TGF-beta agents for various therapies, including long lasting respiratory disease. Examples of anti-TGF-beta agents include TGF-β inhibitors such as antisense oligonucleotides, artemisinin, pharmaceutically acceptable salts forms, esters, polymorphs or stereoisomers thereof, as well as combinations thereof.

In general, the pathology of severe or long-lasting respiratory disease is unpredictable, therefore new therapies will require clinical studies for distinct patient populations. This disclosure provides results for a highly reliable, double-blind, randomized, placebo-controlled study of patients with severe respiratory disease.

In further aspects, this disclosure provides highly stable formulations of anti-TGF-beta agents for therapies for age disease. The stable formulations of this invention provide surprisingly improved clinical results. Stable formulations of agents for suppressing TGF-β can be used to reduce damage to various organs, relieve disease action, and reduce long term post-infection symptoms for long lasting disease.

Methods and compositions of this invention can be used for inhibiting or suppressing factors in the unpredictable pathology of age disease. In certain embodiments, this disclosure provides methods and compositions for inhibiting the activity of TGF-β and/or suppressing TGF-β related pathologies, which can improve the efficacy for treating or ameliorating the symptoms of long lasting COVID-like and other respiratory diseases.

Compositions and formulations of this disclosure can be used for inhibiting and/or suppressing TGF-β to provide positive clinical results for treating severe and/or long lasting infectious viral disease, as well as other respiratory diseases.

### Methods and Compositions for Age Disease

Methods of this invention include processes for treating or ameliorating the symptoms of age disease in a human subject or animal in need. Such processes can be carried out by preparing a pharmaceutical composition including an agent for inhibiting or suppressing expression of TGF-β, and administering a therapeutically sufficient amount of the composition to the subject.

In some embodiments, this disclosure provides uses of a composition of an agent for inhibiting or suppressing expression of TGF-β for treating or ameliorating the symptoms of age disease in a human subject or animal.

In further embodiments, this disclosure provides uses of a composition of an agent for inhibiting or suppressing expression of TGF-β in the preparation of a medicament for treating or ameliorating the symptoms of age disease in a human subject or animal.

In processes or uses of this invention, the age disease may be due to a viral disease. For example, a viral disease may be caused by SARS, MERS, Coronavirus, HIV, Ebola, Cytomegalovirus, human herpes virus type 6, herpes simplex virus HSV-1, herpes simplex virus HSV-2, Epstein-Barr virus, Hepatitis B virus, Enterovirus D68, or Influenza A. In some embodiments, the age disease may be due to COVID-19, long term coronavirus disease, hyperinflammatory immune disease, severe respiratory disease due to viral infection, age-related fibrotic disease, or age-related cancer.

This invention provides methods and formulations for subjects having age disease who may be hospitalized. The hospitalization of a subject can be due to any one of the following:
WHO COVID-19 Clinical Improvement Ordinal Scale Criteria 3, wherein the subject is hospitalized without oxygen therapy;
WHO COVID-19 Clinical Improvement Ordinal Scale Criteria 4, wherein the subject is hospitalized with oxygen by mask or nasal prongs;
WHO COVID-19 Clinical Improvement Ordinal Scale Criteria 5, wherein the subject is hospitalized with non-invasive mechanical ventilation or high-flow oxygen; and
WHO COVID-19 Clinical Improvement Ordinal Scale Criteria 6, wherein the subject is hospitalized with intubation and mechanical ventilation.

A subject of this disclosure who is hospitalized may have age greater than 60 years and may be hospitalized and presenting at least one medical risk factor selected from:
absolute lymphocyte count ≤1000 cells/mm³;
age≥60 years;
hypertension;
diabetes;
cardiac failure; and
COPD.

In further embodiments, the processes or uses of this invention can be applied where subjects have age greater than 35 years and are hospitalized and exhibiting low PaO₂ less than 76 or 77 mmHg.

In additional embodiments, the disease can be multiorgan fibrosis due to aging including any one of lung failure, cardiac failure, kidney failure, and brain cognitive dysfunction.

In certain embodiments, subjects may have long term COVID disease symptoms due to any COVID variant.

In further aspects, the processes and/or uses of this invention can be combined or applied with a standard of care treatment recognized for the disease. Examples of additional medicaments that may be used include anti-inflammatories, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

In further aspects, the processes and/or uses of this invention can be combined or applied along with anti-inflammatory medications. In certain embodiments, the processes and/or uses of this invention can be combined or applied along with anti-inflammatory steroid medications.

In additional embodiments, the processes or uses of this invention can achieve surprisingly improved outcomes. A subject upon administration or use of a composition of this disclosure may have an improved clinical score based on an eight point WHO COVID-19 Clinical Improvement Ordinal Scale at Day 14, or Day 28.

In further embodiments, the processes or uses of this invention can achieve surprisingly improved subject symptoms. A subject upon administration or use of a composition of this disclosure may have an improved level of an inflammatory biomarker. Examples of inflammatory markers include C reactive protein, erythrocyte sedimentation rate, procalcitonin level, plasma viscosity, and fibrinogen level.

In additional embodiments, the processes or uses of this invention can achieve surprisingly improved outcomes. A subject upon administration or use of a composition of this disclosure may have a decreased mortality rate at Day 7, or Day 14, or Day 28.

In certain embodiments, the processes or uses of this invention can achieve surprisingly improved clinical outcomes. A subject upon administration or use of a composition of this disclosure may have increased viral load knockdown at Day 7, or Day 14, or Day 28.

In additional embodiments, the processes or uses of this invention can achieve surprisingly improved outcomes. A subject upon administration or use of a composition of this disclosure may have increased survival rate at Day 14, or Day 28.

Examples of agents of this disclosure for inhibiting or suppressing expression of TGF-β include antisense oligonucleotides specific for TGF-β1, TGF-β2, or TGF-β3.

Examples of agents of this disclosure for inhibiting or suppressing expression of TGF-β include TGF-β2-specific antisense oligonucleotides given in SEQ ID NOs:1-9 herein.

SEQ ID NO: 1
gtaggtaaaa acctaatat.
SEQ ID NO: 2
gttcgtttag agaacagatc.
SEQ ID NO: 3
taaagttcgt ttagagaaca g.
SEQ ID NO: 4
agccctgtat acgac.
SEQ ID NO: 5
gtaggtaaaa acctaatat.
SEQ ID NO: 6
cgtttagaga acagatctac.
SEQ ID NO: 7
cattgtagat gtcaaaagcc.
SEQ ID NO: 8
ctccctcatg gtggcagttg a.
SEQ ID NO: 9
cggcatgtct attttgta.

Antisense oligonucleotides given in SEQ ID NOs:1-9 herein can be chemically-modified, as known in the art.

Examples of agents of this disclosure for inhibiting or suppressing expression of TGF-β include artemisinin extracts, a pharmaceutically-acceptable salt, salt polymorph, ester, or isomer thereof, and any combination thereof. In some embodiments, this disclosure includes a substantially pure artemisinin having a purity of at least 60%, or 70%, or 80%, or 90%, or 95%.

In certain embodiments, agents of this disclosure for inhibiting or suppressing expression of TGF-β may be prepared from a lyophilized powder of the agent.

More specifically, an agent may be a TGF-β2-specific antisense oligonucleotide selected from SEQ ID NOs:1-9, and administered or used by continuous intravenous infusion at a dose of 140 mg/m 2 on Days 1 to 7, or at a dose of 1000 mg/m 2 on Days 1 to 7, or at a dose of 180 mg/m 2 on Days 1 to 7, or at a dose of 200 mg/m 2 on Days 1 to 7.

In some embodiments, an agent may be a TGF-β2-specific antisense oligonucleotide selected from SEQ ID NOs:1-9, and chemically-modified variants thereof, and administered or used by continuous intravenous infusion with a Cmax value of from 2 to 3 μg/mL.

In further embodiments, an agent may be a TGF-β2-specific antisense oligonucleotide selected from SEQ ID NOs:1-9 and chemically-modified variants thereof, and administered or used by continuous intravenous infusion at a dose of 140 mg/m 2 on Days 1 to 7, either singly or in combination with artemisinin in any form at a dose of 500 mg per day on Days 1 to 5.

Examples of agents of this disclosure for inhibiting TGF-β include agents for specifically inhibiting TGF-β1, TGF-β2, or TGF-β3.

Embodiments of this invention involving administration or use of a composition of an agent can ameliorate or suppress symptoms due to TGF-β induced proteins.

Embodiments of this invention involving administration or use of a composition of an agent can ameliorate or suppress symptoms due to long lasting respiratory coronavirus infection.

The agent for inhibiting or suppressing expression of TGF-β may be an artemisinin formulation, comprising 90-95% pure artemisinin extract, or a pharmaceutically-acceptable salt, salt polymorph, ester, or isomer thereof, and one or more pharmaceutically acceptable excipients. Excipients may comprise any one or more pharmaceutically acceptable excipients selected from diluents, stabilizers, disintegrants and anticaking agents. In some embodiments, the excipients may comprise any one or more of microcrystalline cellulose, polysorbate 80, crospovidone, croscarmellose sodium, and magnesium stearate.

In further embodiments, the agent for inhibiting or suppressing expression of TGF-β can be an artemisinin compound or derivative thereof, or a pharmaceutically-acceptable salt, salt polymorph, ester, or isomer thereof.

As used herein, a derivative encompasses chemical modifications that provide structural analogs of a compound. For example, substituents or substitutions of an alkyl group can provide structural analogs.

Embodiments of this invention include processes or uses wherein the agent for inhibiting or suppressing expression of TGF-β is a compound, or ligand comprising a small molecule or polypeptide, that interacts with Site I of TGF-β comprising Trp30 and/or Site II of TGF-β comprising Arg15, Gln19, and Phe8, or a pharmaceutically-acceptable salt, salt polymorph, ester, or isomer thereof.

In some embodiments, the agent for inhibiting or suppressing expression of TGF-β may be a polypeptide or peptide mimetic of Site I of TGF-β comprising residues Phe24-Lys37 and/or Site II of TGF-β comprising residues Cys7-G1n19, or a pharmaceutically-acceptable salt, salt polymorph, ester, or isomer thereof.

In further embodiments, the agent for inhibiting or suppressing expression of TGF-β may be an antibody or antibody fragment, humanized or non-humanized, with affinity for Site I of TGF-β comprising residues Phe24-Lys37 and/or Site II of TGF-β comprising residues Cys7-G1n19.

In additional embodiments, the agent for inhibiting or suppressing expression of TGF-β may be a compound comprising a sesquiterprene lactone or derivative thereof, or a pharmaceutically-acceptable salt, salt polymorph, ester, or isomer thereof.

In certain embodiments, the agent for inhibiting or suppressing expression of TGF-β may be a compound comprising three isoprenyl groups and one lactone ring, or derivative thereof, or a pharmaceutically-acceptable salt, salt polymorph, ester, or isomer thereof.

In further embodiments, the process or use may suppress symptoms due to any one of long term coronavirus disease, hyperinflammatory immune disease, severe respiratory disease due to viral infection, age-related fibrotic disease, or age-related cancer upon administration or use of the composition.

In additional embodiments, administration or use of a composition of an agent can ameliorate or suppress symptoms due to multiorgan inflammatory syndrome, cytokine storm, vasculitis, or Kawasaki syndrome.

In various embodiments, the processes or uses of this invention can achieve surprisingly improved outcomes. A subject upon administration or use of a composition of this disclosure may have reduced or suppressed symptoms due to cytokine storm.

In certain embodiments, the processes or uses of this invention can achieve surprisingly improved outcomes. A subject upon administration or use of a composition of this disclosure may have reduced intensive care unit duration.

In further embodiments, the processes or uses of this invention can achieve surprisingly improved outcomes. A subject upon administration or use of a composition of this disclosure may have reduced hospitalization duration.

In additional embodiments, the processes or uses of this invention can achieve surprisingly improved outcomes. A subject upon administration or use of a composition of this disclosure may have increased ventilator-free days.

Embodiments of this invention further include pharmaceutical compositions for inhibiting or suppressing expression of TGF-β, or for inhibiting or suppressing entry or replication of a virus in a cell, or for inhibiting or suppressing an inflammatory response or cytokine storm, or for treating or ameliorating the symptoms of age disease in a human or animal. The pharmaceutical compositions may contain a TGF-β inhibitor, artemisinin, pharmaceutically acceptable salts forms, esters, polymorphs or stereoisomers thereof, and any combination thereof, as well as a carrier. The TGF-β inhibitor may be selected from TGF-β2-specific antisense oligonucleotides SEQ ID NOs:1-9 and chemically-modified variants thereof. The carrier may be sterile water for injection, saline, isotonic saline, or a combination thereof.

Importantly, a composition of this disclosure may be substantially free of excipients. Compositions of this invention which are substantially free of excipients have been found to be surprisingly stable in a carrier. In some embodiments, the composition may be stable for at least 14 days, or at least 21 days, or at least 28 days in a carrier at 37° C. In additional embodiments, a pharmaceutical composition for infusion may contain less than 1% by weight of excipients, or less than 0.5% by weight of excipients, or less than 0.1% by weight of excipients.

Embodiments of this invention further contemplate therapeutic modalities in which a composition of this invention is administered or utilized in combination with a standard of care therapy for the disease. Examples of additional medicaments which may be administered or utilized in combination with a composition of this invention include anti-inflammatories, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

Examples of earlier conditions include viral diseases. An age disease may be due to SARS, MERS, Coronavirus, HIV, Ebola, Cytomegalovirus, human herpes virus type 6, herpes simplex virus HSV-1, herpes simplex virus HSV-2, Epstein-Barr virus, Hepatitis B virus, Enterovirus D68, influenza, or Influenza A.

This invention further provides kits comprising a lyophilized powder in a vial at a content of 250 mg each of one or more TGF-β2-specific antisense oligonucleotides selected from SEQ ID NOs:1-9.

This invention also provides kits comprising a lyophilized powder in a vial at a content of 500 mg of artemisinin or a derivative thereof, or a compound, or ligand comprising a small molecule or polypeptide, that interacts with Site II of TGF-β comprising Arg15, Gln19, and Phe8, a sesquiterprene lactone or derivative thereof, or a compound comprising three isoprenyl groups and one lactone ring and derivatives thereof, or a pharmaceutically-acceptable salt, salt polymorph, ester, or isomer thereof, or any combination of the foregoing.

### Therapeutic Strategy

This invention describes compositions and methods for treating or ameliorating symptoms of age and age related disease including cancer, fibrosis, and hyperinflammatory response.

Without wishing to be bound by theory, COVID-19 survivors have greater than 3× increase in risk of respiratory conditions months after recovery from COVID-19, including respiratory failure, insufficiency, arrest and lower respiratory disease. Thus, long lasting coronavirus infection and failure of conventional treatments for COVID-19 suggest that the therapeutic strategy and target was not entirely accurate.

For example, a surge may be the underlying cause of COVID-19 pathology. COVID-19 disease results in massive release of TGF-β which is ultimately responsible for fibrosis and organ scarring related to post recovery symptoms observed in long term COVID-19 disease.

TGF-β may also be involved in the pathogenesis of lung tissue remodeling and lung fibrosis that follows acute respiratory distress syndrome (ARDS). Specifically, TGF-β contributes to the development of lung fibrosis by stimulating the proliferation/differentiation of lung fibroblasts, accumulation of collagen and other extracellular matrix proteins in the pulmonary interstitial and alveolar space, leading to the occurrence and development of pulmonary fibrosis. Lung TGF-β1 mRNA expression is higher in SARS-CoV-2 infected patients than controls. Similarly, TGF-β protein is higher in lung of COVID-19 patients. Serum levels of TGF-β1 protein can be increased significantly in COVID-19. Post COVID-19 patients with lung fibrosis-like symptoms may have higher levels of IL-1α and TGF-β but lower levels of IFN-β, unlike vaccinated subjects and post COVID-19 patients who did not show fibrosis-like signs.

Severe COVID-19 cases may have increased incidence of adverse cardiac remodeling and kidney remodeling because of increased TGF-β/Smad3 signaling causing tubular epithelial cell death. Activation of TGF-β signaling and oxidative overload may result in tau hyperphosphorylation typically associated with cognitive dysfunction or impairment, such as Alzheimer/Dementia.

This invention provides therapeutics for age disease, caused by TGF-β and long term diseases. This therapeutic strategy is valid because viral replication cannot occur without TGF-β. Processes and uses of antisense oligonucleotides such as OT-101 have been shown for suppressing both SARS-CoV-1 and SARS-CoV-2 replication on Vero cells with EC50 comparable to remdesivir (EC50 of 330 nM vs. 770 nM) and selectivity (SI) is higher than the SI of remdesivir (>500 vs. >130). Moreover, artemisinin, purified from herb Artemisia annua, inhibits TGF-β activity and neutralizes SARS-CoV-2 (COVID-19) in vitro at an EC50 of 0.45 ug/ml and a Safety Index of 140, which is better than remdesivir and chloroquine.

This therapeutic strategy is valid because it can stop viral infection and prevent scarring and long term symptoms and effects.

This therapeutic strategy is also valid because a TGF-β surge and cytokine storm cannot occur without TGF-β. Thus, severe and long lasting symptoms due to a TGF-β surge, including ARDS and cytokine storm, are suppressed by targeting TGF-β and/or OT-101. For example, TGF-β knockout mice that are genetically TGF-β deficient are resistant to the influenza virus due to absence of TGF-β surge.

This therapeutic strategy is further valid because it targets host protein TGF-β that plays a pivotal role in ARDS pathophysiology and not a virus-intrinsic target. Therefore processes and uses for OT-101 and/or artemisinin should not promote drug-resistant viral mutations.

OT-101 is an antisense oligonucleotide against TGF-β2 and artemisinin is a small molecule inhibitor of TGF-β, which have been shown to be clinical effective against COVID.

For example, OT-101 was used in intravenous ClinicalTrials.gov identifier: NCT00844064, hereby incorporated by reference.

In this invention, clinical effectiveness of these agents is demonstrated against TGF-β mediate scarring and/or fibrosis, long term symptoms, and age disease.

### Antisense Oligonucleotides for COVID-19

This invention describes compositions and methods for using TGF-β as a valid target for the treatment of COVID-19, and methods for treating COVID-19 with TGF-β inhibition.

An antisense oligonucleotide (ASO) can be a single-stranded deoxyribonucleotide, which may be complementary to an mRNA target. The antisense therapy may downregulate a molecular target, which may be achieved by induction of RNase H endonuclease activity that cleaves the RNA-DNA heteroduplex with a significant reduction of the target gene translation. Other ASO mechanisms can include inhibition of 5′ cap formation, alteration of splicing process such as splice-switching, and steric hindrance of ribosomal activity.

Antisense therapeutic strategies can utilize single-stranded DNA oligonucleotides that inhibit protein production by mediating the catalytic degradation of a target mRNA, or by binding to sites on mRNA needed for translation. Antisense oligonucleotides can be designed to target the viral RNA genome or viral transcripts. Antisense oligonucleotides can provide an approach for identifying potential targets, and therefore represent potential therapeutics.

Antisense oligonucleotides can be small synthetic pieces of single-stranded DNA that may be 15-30 nucleotides in length. An ASO may specifically bind to a complementary DNA/RNA sequence by Watson-Crick hybridization and once bound to the target RNA, inhibit the translational processes either by inducing cleavage mechanisms or by inhibiting mRNA maturation. An ASO may selectively inhibit gene expression with specificity. Chemical modifications of DNA or RNA can be used to increase stability.

For example, modifications can be introduced in the phosphodiester bond, the sugar ring, and the backbone. ASO antiviral agents may block translational processes either by (i) ribonuclease H (RNAse H) or RNase P mediated cleavage of mRNA or (ii) by sterically (non-bonding) blocking enzymes that are involved in the target gene translation.

Without wishing to be bound by theory, SARS-CoV PLpro may significantly induce TGF-(3-mediated pro-fibrotic response via a ROS/p38 MAPK/STAT3/Egr-1 pathway in vitro and in vivo. PLpro may also trigger Egr-1 dependent transcription of TSP-1 as an important role in latent TGF-β1 activation. Blocking TGF-β may inhibit or reduce the complication of viral spread and fibrosis as well as giving chance for cellular immunity to exert its effect and hence reduce viral yield in the infected cells. Knockdown of TGF-β gene expression may inhibit replication of PRRSV and also improve immune responsiveness following viral infection. Epithelial cells can have a role in orchestrating pulmonary homeostasis and defense against pathogens. TGF-β can regulate an array of immune responses, both inflammatory and regulatory, however, its function is highly location and context dependent. Epithelial derived TGF-β may act as a pro-viral factor suppressing early immune responses during influenza A infection. Mice specifically lacking bronchial epithelial TGF-b1 (epTGFbKO) can display marked protection from influenza-induced weight loss, airway inflammation, and pathology. However, protection from influenza-induced pathology may not be associated with a heightened lymphocytic immune response. The kinetics of interferon beta (IFNb) release into the airways may be significantly enhanced in epTGFbKO mice as compared to control mice, with elevated IFNb on day 1 in epTGFbKO as compared to control mice. This may have induced a heighted antiviral state resulting in impaired viral replication in epTGFbKO mice. TGF-β suppression may act against viral infection. The TGF-β storm driven by viral infection of epithelial cells and multiplication may be the source of TGF-β. Sudden and uncontrolled increases in active TGF-β, possibly with the help of some proinflammatory cytokines such as TNFα, IL-6, and IL-1β, may inevitably result in rapid and massive edema and fibrosis that remodels and ultimately blocks airways. This may lead to the functional failure of the lungs and morbidity of the patients.

### Pathology of Storm Disease

Without wishing to be bound by theory, coronavirus entry into cells can be followed by suppression of cellular replication and redirection of cellular machineries to the replication of the virus. SARS-CoV-1 infection of VeroE6 cells may inhibit cell proliferation by both the phosphatidylinositol 3′-kinase/Akt signaling pathway and by apoptosis. The nucleocapsid protein of SARS-CoV-1 can inhibit the cyclin-dependent kinase complex and block S phase progression in mammalian cells including VeroE6. Further, SARS-CoV-1 7a protein may block cell cycle progression at G0/G1 phase via the cyclin D3/pRB pathway of HEK293, COS-7, and Vero cells. Murine coronavirus replication can induce cell cycle arrest in G0/G1 phase in infected 17C1-1 cells through reduction in Cdk activities and pRb phosphorylation. Infection of asynchronous replicating and synchronized replicating cells with the avian coronavirus infectious bronchitis virus (IBV) can arrest infected cells in the G1/M phase of the cell cycle. Cell cycle arrest may also be centrally mediated by up-regulation of TGF-β. SARS coronavirus can upregulate TGF-β via its nucleocapsid protein and papain-like protease (PLpro). SARS coronavirus PLpro can activate TGF-β1 transcription both in cell-based assay and in mouse model with direct pulmonary injection. TGF-β overexpression in SARS patients lung samples may occur. Suppression of TGF-β expression by OT-101 may have suppressed SARS-CoV1 and SARS-CoV2 replication in viral replication assays. Artemisinin, a TGF-β inhibitor, may also suppress SARS-CoV2 replication. Induction of TGF-β following infection may result in cell cycle arrest to allow for diversion of cellular machinery to viral production. Viral load may increase with a proportional increase in TGF-β, which in turn can drive the progression of COVID-19 disease. Viral load can be closely associated with drastically elevated IL-6 level in critically ill COVID-19 patients. By targeting TGF-β, OT-101 can shut off the engine behind COVID-19 and allow patients to recover without going into respiratory crisis.

All publications including patents, patent application publications, and non-patent publications referred to in this description, as well as the sequence listing are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

The terms “a,” “an,” “the,” and similar terms describing the invention, and in the claims, are to be construed to include both the singular and the plural.

### EXAMPLES

Example 1. Preparation of stable drug agent solutions free of excipients for suppressing TGF-β. This example demonstrates preparation of a stable and compatible solutions of antisense agents for suppressing and inhibiting TGF-β that are substantially free of excipients.

Experiments set forth below showed that a OT-101 solution of 10 μM (61.43 μg/mL) in NaCl at 5° C. and 37° C. was surprisingly stable for at least two weeks. Further, OT-101 solutions of 7.35 mg/mL and 25 mg/mL in isotonic saline at 5° C. and 37° C. were surprisingly stable for at least two weeks.

In-use conditions mimicking clinical studies and the outcomes of the studies are shown in Table 1 and Table 2. Table 1 shows results for an antisense oligonucleotide against TGF-β for administration to patients by IV infusion.

TABLE 1
In-use stability study of antisense oligonucleotide against TGF-β

| In-use Conditions | Outcome of the Study |
|---|---|
| Drug solution: 10 μM (61.43 μg/mL) OT-101 in | The Drug Delivery System used in |
| isotonic saline | Clinical Study AP 12009-G005 |
| Flow rate: 4 μL/min (corresponds to 5.76 mL/day) | was suitable for its intended use |
| Storage of the pump (including drug reservoir) and | with regard to the compatibility |
| the non implanted parts of the drug delivery system at | with a 10 μM AP 12009 Drug |
| ambient temperature | Solution |
| Storage of the implanted parts of the drug delivery | |
| system at 37° C. | |
| Drug reservoir content: 50 mL | |
| Duration of test: 8 days | |
| The conditions are same as above (QC-AP0132R) | Based on the in-use study results, |
| except the external component of the Drug Delivery | the Drug Delivery System for |
| System kept at 30° C. to mimic the clincally relevant | Clinical Study AP12009-G005 was |
| Climatic Zone III/IV | considered suitable for its intended |
| | use in Climatic Zone III/IV |
| Drug Conc.: 10 μM (61.43 μg/mL). | Based on the results AP 12009 |
| Temp. 5° C. and 37° C., Diluent 0.9% NaCl, Container | (OT-101) of 10 μM in NaCl at 5° C. |
| 6 R Sample Vials, Duration 2 weeks | and 37° C. and AP 12009 (OT-101) |
| Drug Conc.: 1 mg/mL | solution in WFI at 5° C. are stable |
| Temp. 5° C., Diluent WFI, Container 6 R Sample | for at least two weeks |
| Vials, Duration 2 weeks | |
| Drug solution: 15 mg/mL (calculated based on a mean | None of the components tested had |
| dosage of 195 mg/m²/d and mean body surface of | impact on the quality of the |
| 1.85 m²) | delivered Drug Solution under in- |
| Flow rate: 1 mL/h (corresponds to 24 mL/day) | use conditions. Based on the result |
| Storage of the pump (including drug reservoir) and | all Drug Delivery Systems |
| the non implanted parts of the drug delivery system at | composed of any combination of |
| 30° C. | one of the tested pumps are |
| Storage of the implanted parts of the drug delivery | considered suitable with regard to |
| system at 37° C. | their compatibility with OT-10 |
| Drug reservoir content: 120 mL | Drug solution |
| Duration of test: 5 days | |

The experiments of Table 1 show that antisense oligonucleotide OT-101 at 10 μM in NaCl at 5° C. and 37° C. was surprisingly stable for at least two weeks. The experiments of Table 1 show that antisense oligonucleotide OT-101 in WFI at 5° C. was stable for at least two weeks.

Table 2 shows results for an antisense oligonucleotide against TGF-β for administration to patients by IV infusion.

TABLE 2
In-use stability study of TGF-β inhibitor trabedersen

| In-use Conditions | Outcome of the Study |
|---|---|
| Drug Conc.: 7.35 mg/mL | Based on the results from the study |
| and 25 mg/mL | concentrated trabedersen (AP |
| Temp. 5° C. and 37° C. | 12009) solutions in NaCl was |
| Diluent 0.9% NaCl | stable for at least two weeks |
| Container 6 R Sample Vials, | |
| Duration 2 weeks | |
| Drug Conc.: 18.23 mg/mL | The results of this study |
| Temp. 20-25° C. | demonstrate that the Cadd |
| Diluent 0.9% NaCl | Medication Cassette Reservoir |
| Container: Cadd Medication | warrants sterility of a sterile filled |
| Casette Resorvoir | drug solution for a period of at least |
| Duration: 7 days | 7 days. |

The experiments of Table 2 show that TGF-β antisense oligonucleotide trabedersen at 7.35 mg/mL and 25 mg/mL in NaCl at 5° C. and 37° C. was surprisingly stable for at least two weeks.

A further in-use stability study of OT-101 at 10 μM (61.43 μg/mL) was performed. An analytical stock solution of concentration 1.0 mg/mL and 10 μM (61.43 μg/mL) was used. A 10 μM (61.43 μg/mL) OT-101 clinically relevant concentration in 0.9% NaCl was checked for stability after storage at 5° C. and 37° C., and a 1 mg/mL OT-101 analytical stock solution in Water for Injection was checked for stability after storage at 5° C. for two weeks. The materials used for the experiments are shown in Table 3.

TABLE 3
Materials and drug solution

| Description | Manufacturer | Ref. No. | Lot. No. |
|---|---|---|---|
| OT-101 Working | Avecia, USA | — | AQX-05L-002 |
| Standard | | | Anal A 01/01 |
| 0.9% NaCL | Braun, Germany | 3820084 | 9152A91 |
| solution | | | |
| Ampuwa Water | Fresenius | 40676.00.00 | 14DD1005 |
| for Injection | | | |
| 6 R Sample Vials | PharMediPack, | 05000613100 | 20081216 |
| | Germany | | |
| Fluorotec Stoppers | West Phar., USA | 12414110/40 | 1092007924 |
| | | grey | |
| Alu Caps | PharMediPack, | 03500103000 | 0507010302 |
| | Germany | | |

The impurity profiles of the samples were determined by RP-HPLC and the concentrations were determined by UV-spectrometry. The impurities profile of the samples by RP-HPLC are shown in Table 4.

TABLE 4
Reverse Phase HPLC Samples for 10 μM (61.43 μg/mL)
Solutions

| | | | | | | Total |
|---|---|---|---|---|---|---|
| | AP | 3′N- | 3′N- | 5′N1/ | | Other |
| | 12009 | 2* | 1 * | PO* | CNET* | Imp |
| Acceptance | ≥85 | ≤0.6 | ≤3.4 | ≤7.6 | ≤5.2 | Report |
| Criteria | | | | | | |
| (AP 12009 | | | | | | |
| Drug Product) | | | | | | |
| Description of | | | | | | |
| Sample | | | | | | |
| 10 μM in saline, | 96.61 | n.d. | 0.96 | 1.36 | 0.40 | 0.70 |
| t = 0 d −20° C. | | | | | | |
| 10 μM in saline, | 96.76 | n.d. | 0.93 | 1.30 | 0.38 | 0.64 |
| t = 7 d 5° C. | | | | | | |
| 10 μM in saline, | 96.61 | n.d. | 1.00 | 1.37 | 0.37 | 0.67 |
| t = 14 d 5° C. | | | | | | |
| 10 μM in saline, | 96.29 | n.d. | 1.08 | 1.55 | 0.36 | 0.73 |
| t = 7 d 37° C. | | | | | | |
| 10 μM in saline, | 96.09 | n.d. | 0.99 | 1.75 | 0.41 | 0.76 |
| t = 14 d 37° C. | | | | | | |

*PO = impurity with one phosphorothioate moiety replaced by phosphate moiety (coeluting with 5′N-1)
CNET = impurity with a cynoethyl-moiety added to one of the thymidine nucleotide
3′N-2-impurity missing two 3′-terminal nucleotide
3′N-1-impurity missing the 3′-terminal nucleotide
5′N-1 = impurity missing 5′-terminal nucleotide (coeluting with PO)
n.d. = not detected

The concentration of the samples were compared to the concentration of the Reference Samples (t=0 d). The data are summarized in Table 5. The results are considered to be adequate, when the concentration was between 95 and 105% of the concentration of the respective Reference Sample.

TABLE 5
UV-Analysis of OT-101 Solution of
10 μM (61.43 μg/mL) in 0.9% NaCl

| Sample ID | A_{260 nm} | Concentration (%) |
|---|---|---|
| 10 μM in saline, t = 0 d −20° C. | 0.5291 | 100.0 |
| 10 μM in saline, t = 7 d 5° C. | 0.5321 | 100.6 |
| 10 μM in saline, t = 14 d 5° C. | 0.5282 | 99.8 |
| 10 μM in saline, t = 7 d 37° C. | 0.5326 | 100.7 |
| 10 μM in saline, t = 14 d 37° C. | 0.5288 | 99.9 |

All UV spectra corresponded to the characteristic UV spectrum of OT-101 and the concentrations of all solutions were within a range of ±0.8% of the concentration of the reference (t=0 d). This experiment demonstrated that the concentrations of a 10 μM (61.43 μg/mL) OT-101 solution in isotonic saline after storage at 5° C. and 37° C. for two weeks were substantially unchanged.

These experiments showed that based on the above RP-HPLC impurity levels, UV spectra and concentration profiles, the OT-101 antisense oligonucleotide solutions of 10 μM in NaCl at 5° C. and 37° C. were surprisingly stable for at least two weeks.

A further in-use stability study of OT-101 at 7.35 mg/mL and 25 mg/mL was performed. OT-101 solutions of concentrations 7.35 mg/mL and 25 mg/mL in 0.9% NaCl were checked for stability after storage at 5° C. and 37° C. for two weeks. The materials used for the experiments are shown in Table 6.

TABLE 6
Materials and drug solution

| Description | Manufacturer | Ref. No. | Lot. No. |
|---|---|---|---|
| AP 12009 | Thymoorgan, | — | 08L10AP12009 |
| 250 mg | Germany | | |
| 0.9% NaCl | Braun, Germany | 3820084 | 0214A191 |
| Solution | | | |
| 6 R Sample | PharMediPack, | 05000613100 | 20091188 |
| Vials | Germany | | |
| Fluorotec | West Phar., | 12414110/40 Grey | 1072036272 |
| Telfon | USA | | |
| Stoppers | | | |
| Alu Caps | PharMediPack, | 03500103000 | 0507010302 |
| | Germany | | |

The impurity profiles of the samples were determined by RP-HPLC and the concentrations were determined by UV-spectrometry. The impurities profiles of the samples by RP-HPLC are shown in Table 7.

TABLE 7
Reverse Phase HPLC Samples for 7.35 mg/mL and 25 mg/mL
Solutions

| | | | | | | Total |
|---|---|---|---|---|---|---|
| | AP | | | 5′N-1/ | | Other |
| | 12009 | 3′N-2* | 3′N-1* | PO* | CNET* | Imp |
| Acceptance | ≥85 | ≤0.6 | ≤3.4 | ≤7.6 | ≤5.2 | Report |
| Criteria | | | | | | |
| (AP 12009 | | | | | | |
| Drug Product) | | | | | | |
| Description of | 95.13 | n.d. | 1.14 | 1.78 | 0.47 | 1.48 |
| Sample | | | | | | |
| 7.35 mg/mL, | 95.12 | n.d. | 1.14 | 1.76 | 0.48 | 1.51 |
| t = 0 d −20° C. | | | | | | |
| 7.35 mg/mL, | 95.13 | n.d. | 1.15 | 1.77 | 0.47 | 1.48 |
| t = 7 d 5° C. | | | | | | |
| 7.35 mg/mL, | 94.79 | n.d. | 1.19 | 2.01 | 0.47 | 1.55 |
| t = 14 d 5° C. | | | | | | |
| 7.35 mg/mL, | 94.57 | n.d. | 1.24 | 2.13 | 0.48 | 1.59 |
| t = 7 d 37° C. | | | | | | |
| 7.35 mg/mL, | 95.08 | n.d. | 1.15 | 1.75 | 0.47 | 1.55 |
| t = 14 d 37° C. | | | | | | |
| 25 mg/mL, | 95.04 | n.d. | 1.15 | 1.74 | 0.47 | 1.74 |
| t = 0 d −20° C. | | | | | | |
| 25 mg/mL, | 95.05 | n.d. | 1.13 | 1.77 | 0.47 | 1.57 |
| t = 7 d 5° C. | | | | | | |
| 25 mg/mL, | 94.81 | n.d. | 1.18 | 1.91 | 0.48 | 1.63 |
| t = 14 d 5° C. | | | | | | |
| 25 mg/mL, | 94.62 | n.d. | 1.23 | 1.98 | 0.47 | 1.98 |
| t = 7 d 37° C. | | | | | | |
| 25 mg/mL, | 95.13 | n.d. | 1.14 | 1.78 | 0.47 | 1.48 |
| t = 14 d 37° C. | | | | | | |

*PO = impurity with one phosphorothioate moiety replaced by phosphate moiety (coeluting with 5′N-1)
CNET = impurity with a cynoethyl-moiety added to one of the thymidine nucleotide
3′N-2 = impurity missing two 3′-terminal nucleotide
3′N-1 = impurity missing the 3′-terminal nucleotide
5′N-1 = impurity missing 5′-terminal nucleotide (coeluting with PO)
n.d. = not detected

The impurity profile was adequate for the intended administration.

The concentrations of the samples were compared to the concentrations of the Reference Samples (t=0 d). The data are summarized in Table 8 and Table 9. The results were adequate, when the concentration was between 95 and 105% of the concentration of the respective Reference Sample.

TABLE 8
UV-Analysis of OT-101 Solution of
7.35 mg/mL in 0.9% NaCl Solution

| Sample ID | A_{260 nm} | Concentration (%) |
|---|---|---|
| 7.35 mg/mL, t = 0 d @ −20° C. | 0.4441 | 100.00 |
| 7.35 mg/mL, t = 7 d @ 5° C. | 0.4516 | 101.69 |
| 7.35 mg/mL, t = 14 d @ 5° C. | 0.4534 | 102.09 |
| 7.35 mg/mL, t = 7 d @ 37° C. | 0.4525 | 101.89 |
| 7.35 mg/mL, t = 14 d @ 37° C. | 0.4519 | 101.76 |

TABLE 9
UV-Analysis of OT-101 Solution of
25 mg/mL in 0.9% NaCl

| Sample ID | A_{260 nm} | Concentration (%) |
|---|---|---|
| 25 mg/mL, t = 0 d @ −20° C. | 0.4837 | 100.00 |
| 25 mg/mL, t = 7 d @ 5° C. | 0.4960 | 102.54 |
| 25 mg/mL, t = 14 d @ 5° C. | 0.4949 | 102.32 |
| 25 mg/mL, t = 7 d @ 37° C. | 0.5010 | 103.58 |
| 25 mg/mL, t = 14 d @ 37° C. | 0.4989 | 103.14 |

All UV spectra corresponded to the characteristic UV spectrum of OT-101 and the concentrations of all solutions were within a range of ±3.58% of the concentration of the reference (t=0 d). This experiment demonstrated that the concentrations of 7.35 mg/mL and 25 mg/mL of OT-101 solution in isotonic saline after storage at 5° C. and 37° C. for two weeks are surprisingly stable and unchanged. Based on the above RP-HPLC impurity levels, UV spectra and concentration profiles, the OT-101 solutions of 7.35 mg/mL and 25 mg/mL in isotonic saline solution at 5° C. and 37° C. were surprisingly stable for at least two weeks.

The experiments set forth above further showed that a 15 mg/mL OT-101 Drug Solution in isotonic saline at a flow rate of 1 mL/h over a period of four days was surprisingly stable for the intended Drug Delivery System for IV Infusion.

The experiments set forth above further showed that a 10 μM (61.43 μg/mL) OT-101 Drug Solution in isotonic saline at a flow rate of 0.24 mL/h over a period of seven days was surprisingly stable for the intended Drug Delivery System for IV Infusion.

Example 2. New medical compositions, preparations, and methods discovered for inhibiting TGF-β using primary and alternative binding sites. This example demonstrates identification and use of new medical compositions, preparations, and methods which have been discovered for inhibiting TGF-β. New compositions, preparations, and methods were discovered using bioinformatic structure-based ligand design to identify and measure primary and alternative binding sites of TGF-β1.

Protein crystal structure for TGFβ1 was retrieved from protein data bank (https://www.rcsb.org/) with the accession code 3KFD. The protein was prepared by adding hydrogen atom, removing salts and ion. Missing side chains and loops were added. Finally, proteins were subjected to energy minimization to relax the coordinates. All other parameters were kept default. PocketFinder bioinformatic platform was used to detect primary and alternative binding sites of the protein target. The results were analyzed to identify the structure of binding sites and the orientations of residues neighboring a bound ligand.

A ligand structure based on artemisinin was used for docking calculations with the structure of TGFβ1. Before docking, the test structure was optimized to relax the coordinates. Pocket residues were selected to generate the grid before docking, and a grid was generated for each identified site. Docking of the artemisinin ligand structure was carried out in the generated grid for each target individually. Before docking all parameters were kept default. Ten poses were generated for the docked ligand at each site, and a single final pose was obtained as a result. Each docking output was scored and the ligand conformation determined. The nature and kind of binding interactions for the ligand were determined.

The three dimensional architecture of the protein was mainly composed of beta sheets and long flexible loops. The structure was not tightly packed, so that targeting with small molecules required extensive calculations. Small hydrophobic sub-pockets were formed into which small molecules such as artemisinin could be occupied with the polar side exposed to solvent. Solvent-exposed sites or pockets were detected for which solvent-accessible surface area of the protein was very high.

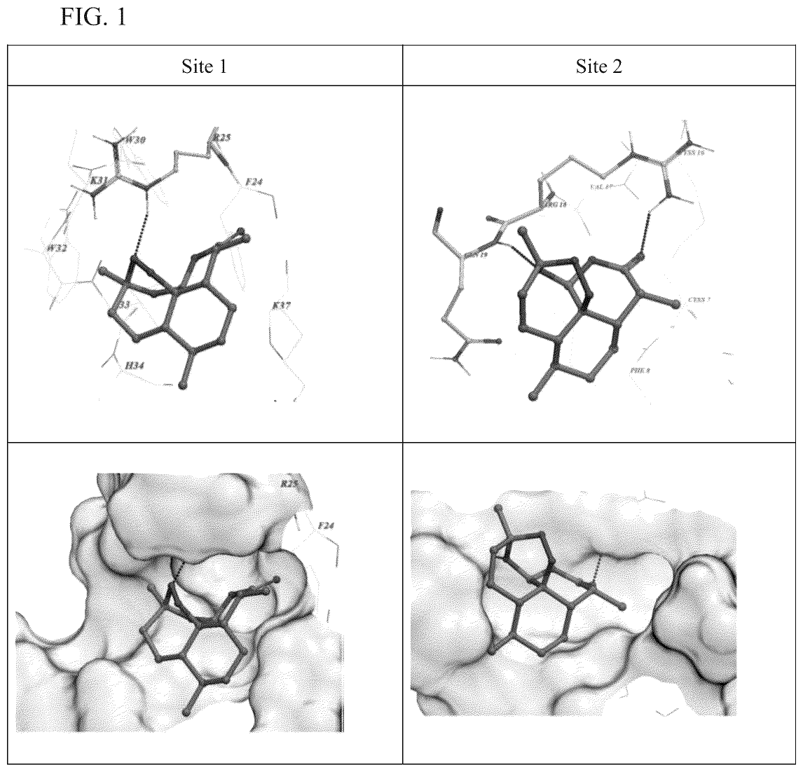

The results determined two sites for binding activity. As shown in FIG. 1, Site 1 included residues Phe24-Lys37 with a docking score of −1.230. Site 2 included residues Cys?-G1n19 with a docking score of −6.01. Site 1 and Site 2 can be used for screening of molecules which will bind into these pockets to block TGF-β activity.

Site II indicated improved ligand sampling inside the pocket for improved binding. The binding interactions of the ligand were within hydrogen bonding distance, which confirmed enzyme-inhibiting activity. Moreover, polar groups of artemisinin occupied deep pocket orientations and confirmed enzyme-inhibiting activity. In particular, the results showed that the keto group of the artemisinin ligand formed a hydrogen bond with the side chain of ARG15. Further, the ether group of the ligand formed a hydrogen bond with the GLN19 backbone NH. A weak hydrophobic interaction was observed between the ligand and PHE8. The core of the pocket was solvent exposed. These structural features confirmed enzyme-inhibiting binding and activity.

New drug agent molecules or ligands which bind to Site 1 or Site 2 have been identified. In some embodiments, artemisinin and its derivatives are agent molecules or ligands which bind to Site 1 or Site 2 and inhibit a TGF-β target, which can include pharmaceutically-acceptable salts, salt polymorphs, esters, or isomers thereof.

In further embodiments, compounds or ligands comprising a small molecule or polypeptide that interacts with Site I of TGF-β comprising Trp30 and/or Site II of TGF-β comprising Arg15, Gln19, and Phe8, or a pharmaceutically-acceptable salt, salt polymorph, ester, or isomer thereof are agent molecules or ligands which bind to Site 1 or Site 2 and inhibit a TGF-β target.

In additional embodiments, polypeptides or peptide mimetics of Site I of TGF-β comprising residues Phe24-Lys37 and/or Site II of TGF-β comprising residues Cys7-G1n19, or a pharmaceutically-acceptable salt, salt polymorph, ester, or isomer thereof are agent molecules or ligands which bind to Site 1 or Site 2 and inhibit a TGF-β target.

In certain embodiments, an antibody or antibody fragment with affinity for Site I of TGF-β comprising residues Phe24-Lys37 and/or Site II of TGF-β comprising residues Cys7-G1n19 are agent molecules or ligands which bind to Site 1 or Site 2 and inhibit a TGF-β target.

In alternative embodiments, compounds comprising a sesquiterprene lactone or derivative thereof, or a pharmaceutically-acceptable salt, salt polymorph, ester, or isomer thereof are agent molecules or ligands which bind to Site 1 or Site 2 and inhibit a TGF-β target.

In further embodiments, compounds comprising three isoprenyl groups and one lactone ring, or derivatives thereof, or a pharmaceutically-acceptable salt, salt polymorph, ester, or isomer thereof are agent molecules or ligands which bind to Site 1 or Site 2 and inhibit a TGF-β target.

Example 3. A Double-Blind, Randomized, Placebo-Controlled, Multi-Center Study of OT-101 in Hospitalized COVID-19 Patients. This example describes a Double-Blind, Randomized, Placebo-Controlled, Multi-Center Study of Antisense Oligonucleotide OT-101 in Hospitalized COVID-19 Patients.

The Primary Objectives were to evaluate the safety and efficacy of OT-101 when used in combination with SoC:
Efficacy: Proportion of subjects with clinical improvement score (measured by an 8 point WHO COVID 19 Clinical Improvement Ordinal Scale) at Day 14; and
Safety: Adverse events, clinical labs, ECG, vital signs, physical exam, radiology tests.

The Secondary Objectives were to further evaluate the efficacy of OT-101 compared to placebo: Odds ratio, ventilator requirements, clinical improvement or worsening, mortality, hospitalization (duration/ICU).

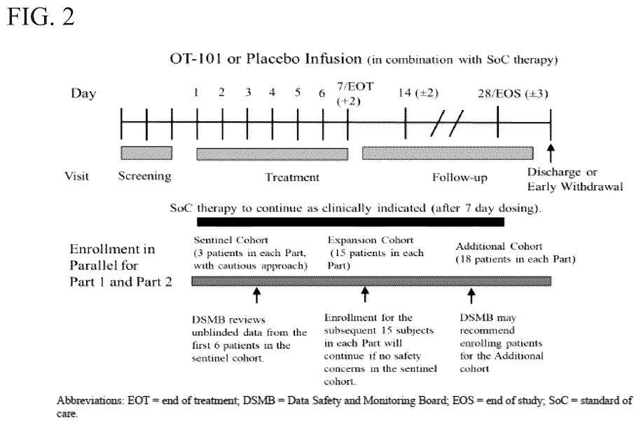

As shown in FIG. 2, the study design was double-blind, randomized, and placebo-controlled. The safety results showed that:
No SAE nor Death nor treatment emergent AE of special interest were observed related to the drug.
Except for the deaths due to secondary infections, lack of standard of care, comorbidity, or high virus load, patients recovered with resolution of COVID symptoms.
No adverse change in vital sign, ECG, physical examination, radiological tests, nor clinical laboratory tests related to drug have been observed. OT-101 was well tolerated in COVID-19 patients with no observed aggravation of cytokine storm such as increase in IL-6 or CRP.
Incidence of adverse events (AEs) and treatment-emergent AEs (TEAEs) were comparable to placebo.
Adverse events leading to premature discontinuation of the study treatment were comparable to placebo.
Treatment emergent AE of special interest (AESIs) (Cytokine release syndrome and thrombocytopenia) was not observed.
No changes in clinical laboratory tests (hematology, clinical chemistry, urinalysis) from baseline.
Comparable changes to placebo in vital sign measurements from baseline.
Comparable changes to placebo in 12 lead electrocardiogram (ECG) from baseline.
Comparable changes to placebo in complete physical examination from baseline.

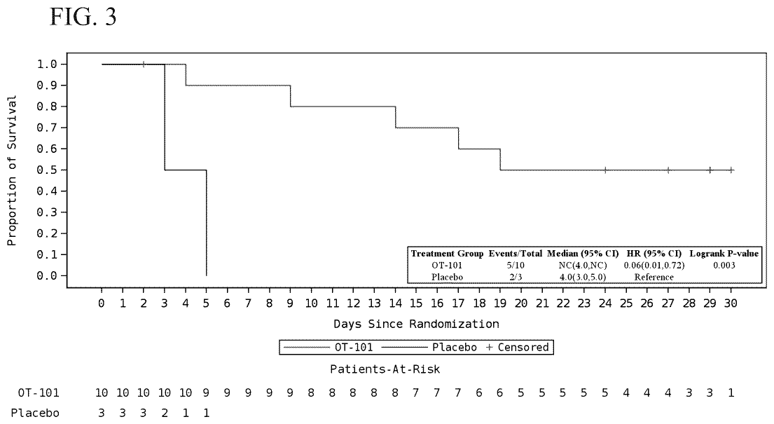

The efficacy results were as follows:
OT-101 treatment was effective in reducing the viral load of all treated patients even those who eventually die suggesting that mortality was due to other causes such as secondary infection.
This was despite the fact that OT-101 subjects were heavily infected with median viral load on D1 at 2.5× higher than that of Placebo subjects.
Viral load was reduced to below detectable level following 7 days of treatment with OT-101.
Viral load reduction greater than 96% on D7 occurred in 17 of 19 patients (89%) for OT-101 versus 6 of 9 patients (67%) for placebo.
As shown in FIG. 3, a surprisingly improved rate of survival was shown in this study as follows:
20 Part 1 Patients and 12 Part 2 Patients, 2:1 Randomization
Day 7 Mortality Rate of 4.5% vs 20% for Placebo for entire population in the trial.
Survival analysis was performed on high risk patients (PaO₂ equal or less than 76 mmHg and age above 35 years)
Overall mortality was 66% in placebo group (2 of 3) versus 50% OT-101 group (5 of 10).
The one patient in OT-101 who died while on treatment had extremely high viral load (73 millions copies/mL by PCR).

This clinical study (C001) was Double-Blind, Randomized, Placebo Controlled, Multi-Center Study of OT-101 in Hospitalized COVID-19 Subjects. This study used the highest standards (randomized, double blind, placebo-controlled) to evaluate the efficacy, safety and tolerability of OT-101 when used in combination with standard of care (SoC) in hospitalized subjects with mild or severe COVID-19.

Part 1 of the study included subjects with mild COVID-19 World Health Organization (WHO) COVID-19 Clinical Improvement Ordinal Scale 3—hospitalized, no oxygen therapy, or 4—hospitalized, oxygen by mask or nasal prongs, and having at least one medical risk factor, such as absolute lymphocyte count, 60 years age, hypertension, diabetes, cardiac failure or chronic obstructive pulmonary disease (COPD) at screening.

Part 2 of the study included subjects with severe COVID-19 WHO COVID-19 Clinical Improvement Ordinal Scale 5—non-invasive ventilation or high flow oxygen, or 6—intubation and mechanical ventilation) at screening.

The Primary Objective was to evaluate the safety and efficacy of OT-101 when used in combination with standard of care (SoC) in hospitalized COVID-19 subjects. The Secondary Objective was to further evaluate the efficacy of OT-101 compared to placebo in hospitalized COVID-19 subjects. Additional Exploratory Objectives were assessment of inflammatory biomarkers and assessment of viral dynamics, using C-reactive protein (CRP), Ferritin, D dimer (comparison of baseline to Day 7, Day 14, Day 28), inflammatory cytokines (interferon gamma (IFN-γ), interleukin 6 (IL-6), tumor necrosis factor (TNF), interleukin 1 (IL-1), interleukin 2 (IL-2), granulocyte macrophage colony stimulating factor (GM-CSF), and complement factors (C3a, C5a, CH50), as well as Change from baseline in biomarkers of Cytokine Release Syndrome (TGF-β 1, 2 & 3 plasma).

The trial enrolled 20 patients of part 1 and 12 patients of part 2. No SAE nor Death nor treatment emergent AE of special interest were observed related to the drug. No adverse change in vital sign, ECG, physical examination, radiological tests, nor clinical laboratory tests related to drug were observed. Antisense OT-101 was well tolerated in COVID-19 patients with no observed aggravation of cytokine storm, such as increased IL-6 or CRP.

Day 7 mortality rate of 4.5% for OT-101 vs 20% for placebo was observed for entire population in the study. In high-risk patients (PaO2 less than 77 mmHg and age above 35 years), overall mortality was 50% in OT-101 group (5 of 10) vs 66% in placebo group (2 of 3) and median overall survival increased from 4 days for Placebo (N=3) to greater than 30 days for OT-101 (N=10, p=0.003).

Antisense OT-101 treatment was effective in reducing the viral load of all treated patients. Viral load reduction greater than 96% on Day 7 occurred in 17 of 19 patients (89%) for OT-101 group versus 6 of 9 patients (67%) for placebo group.

Example 4. TGF-β shuts down cellular replication and promotes viral replication. This experiment showed that suppression of TGF-β expression by OT-101 and Artemisinin suppressed SARS-CoV1 and SARS-CoV2 replication in viral replication assays on Vero 76 cells.

OT-101 activity was superior to antisense specifically designed against SAR-CoV-2 genome along selected 5′-TERM, FS, and LTR. Nonspecific antisense (RSV) did not demonstrate any suppression. The data are shown in the Table 10.

TABLE 10
UV-Analysis of OT-101 Solution of 25 mg/mL in 0.9% NaCl

| Compound | Virus | EC₅₀ | CC₅₀ | SI |
|---|---|---|---|---|
| OT-101 | SARS-CoV-1 (Urbani strain)¹ | 7.6 (1.24 uM) | >1000 | >130 |
| OT-101 | SARS-CoV-1 (Urbani strain)¹ | 26 (4.23 uM) | >1000 | >38 |
| OT-101 | SARS-COV-2 USA_WA1/2020² | 2.0 (0.33 uM) | >1000 | >500 |
| RSV | SARS-COV-2 USA_WA1/2020² | 620.0 | >1000 | >1.6 |
| Artemisinin | SARS-COV-2 USA_WA1/2020² | 0.45 (1.59 uM) | 61.0 | 140 |
| M128533 | SARS-COV-2 USA_WA1/2020² | 0.012 | >10 | >830 |
| Remdesivir | SARS-COV-2 (Wang M, Cao R, | (0.77 uM) | (>100 uM) | >129.87 |
| | Zhang L, et al. (2019-nCOV) in vitro. | | | |
| | Cell Res. 2020; 30(3):269-271) | | | |

RSV- Negative control antisense/M128533- positive control; EC₅₀: 50% effective antiviral concentration (in μg/ml)/CC₅₀: 50% cytotoxic concentration of compound without virus added (in μg/ml)/SI = CC₅₀/EC₅₀;
¹Source of SARS-CoV-1: Centers for Disease Control Stock 809940 (200300592).
²Source of SARS-CoV-2: World Reference Center for Emerging Viruses and Arboviruses (WRCEVA) at UTMB.
Suppression of TGF-β expression by OT-101 or Artemisinin suppressed SARS-CoV1 and SARS-CoV2 replication in the viral replication assays on Vero 76 cells was demonstrated in collaboration with Dr. Brett Hurst at Utah State University, part of the NIAID Antiviral Testing Consortium. In the same study, artemisinin, a reported TGF-β inhibitor (Cao Y, Feng YH, et al., Int. Immunopharmacology, 2019;70:110-116), also suppressed SARS-CoV-2 replication.

Example 5. Artemisinin clinical trial safety and efficacy of Artemisinin on COVID-19. This clinical trial showed Artemisinin was effective against COVID.

The ARTI-19 trial was cleared by Indian regulatory authorities, and was registered under the Clinical Trials Registry India (CTRI). ARTI-19 trial registration information can be found at: CTRI/2020/09/028044. Phase IV study to evaluate the safety and efficacy of Artemisinin on COVID-19 subjects as Interventional. http://ctri.nic.in/Clinicaltrials/advsearch.php. Site specific information is: 1) Government Medical College & Government General Hospital, Srikakulam, ANDHRA PRADESH. 2) Rajarshi Chhatrapati Shahu Maharaj Government Medical College and Chhatrapati Pramila Raje Hospital, MAHARASHTRA. And 3) Seven Star Hospital, MAHARASHTRA. The study was conducted with Abiogenesis India as CRO. A total of 120 patients were randomized into the trial.

The study observed the effect of Artemisinin in COVID-19 patients. These patients had confirmed SARS-CoV-2 infection by RT-PCR and mild and moderate (hospitalized, without oxygen therapy) symptoms of COVID-19. These were patients with scores of 2-4 on the WHO Clinical Progression Scale. Patients were randomized 2:1 into treatment and control group. Group 1—Treatment group: Artemisinin+SOC (Physician's choice). Group 2—Control group: SOC (physician's choice).

ARTI-19 trial of 120 patients of which 80 were on Artemisinin showed a very favorable safety profile, and the only Artemisinin-related adverse events were transient mild rash and mild hypertension. Similarly in the Iranian trial, there was not reported adverse event. Indeed From 1992 to 1997, it was estimated that almost 4 million doses of artemisinin were safely distributed to treat malaria. The standard dosing was 500 mg daily for 5 days (qdx5).

The results of the studies showed that Artemisinin showed a very favorable safety profile, and the only Artemisinin-related adverse events were transient mild rash and mild hypertension. Artemisinin, when added to the SOC, accelerated the recovery of patients with mild-moderate COVID-19 across all COVID-19 symptoms examined including fever, sore throat, dry cough, and ache. Almost all of WHO-4 patients achieved a reduction WHO-3 on the first few doses of Artemisinin, p=0.0043 (n=56, PulmoHeal+SOC vs n=25/SOC). SOC=Standard of care including remdesivir/dex/heparin. Decline in body temperature was faster and higher in Artemisnin+SOC group by day 2 in comparison to SOC arm. Improvement in respiratory rate was faster and higher in Artemisnin+SOC group by day 5 in comparison to SOC arm. All vitals were normalized by the end of the 28 day monitoring period. Most importantly, 02 sat fully recovered by day 28 with Artemisinin+SOC (p<0.0001) but not with SOC alone (p=ns). Similarly, respiratory rate fully recovered by day 28 with Artemisinin+SOC (p<0.0001) but not with SOC alone (ns).

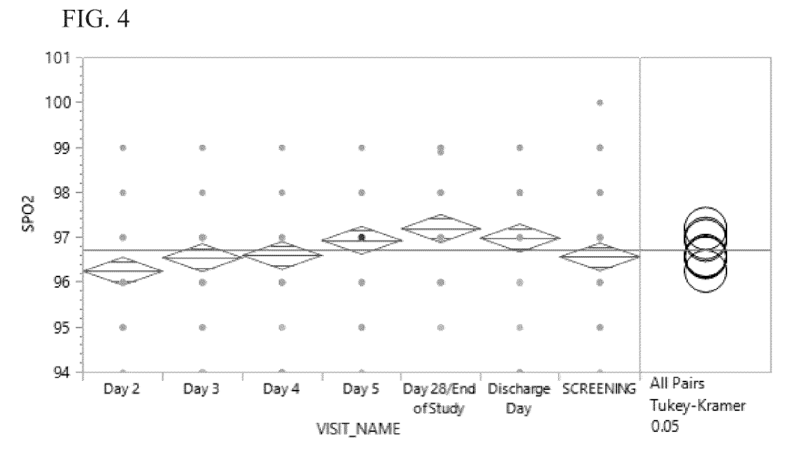

FIG. 4 shows results for such clinical trial using Artemisinin against COVID. Artemisinin restored long term lung functions due to viral infection in patients with confirmed SARS-CoV-2 infection. These results showed that Artemisinin was effective against COVID and provided clinical proof of concept that Artemisinin restores long term lung functions due to viral infection. This surprisingly superior result was consistent with its TGF-β activity, and was estimated to result from inhibition of multiorgan scarring.

This clinical study showed that Artemisinin was active against mild and moderate COVID-19 following the preplanned prospective analysis of ARTI-19 clinical trial, NCT05004753 A Prospective, Randomized, Multi-center, Open label, Interventional Study to Evaluate the Safety and Efficacy of Artemisinin 500 mg capsule in Treatment of Adult Subjects with COVID-19). This clinical study showed that phytomedicine agents against TGF-β can be used effectively for COVID-19 and COVID variants.

This clinical study showed that:
Artemisinin was effective in treating subjects with mild and moderate COVID-19.
Artemisinin+SOC showed significant improvement over SOC in WHO severity scale on Day 4 and Day 5 with p=0.0045 and p=0.0370, respectively.
Artemisinin achieved decline in body temperature surprisingly faster and higher in Artemisnin+SOC group by day 2 in comparison to SOC arm.
Artemisinin showed improvement in respiratory rate faster and higher in Artemisnin+SOC group by day 5 in comparison to SOC arm.
Artemisinin showed improvement in mean SpO2 level significantly higher in Artemisnin+SOC group by day 28 (end of study) in comparison to SOC arm (p=0.029).
Artemisinin showed no clinically significant changes in biochemistry or hematology parameters, and was safe and well-tolerated by the study subjects.

NCT05004753 was a Prospective, Randomized, Multi-center, Open label, Interventional Study to Evaluate the Safety and Efficacy of Artemisinin 500 mg capsule in Treatment of Adult Subjects with COVID-19. This was an open label, prospective, multicenter study. Subjects with a clinical diagnosis of mild to moderate COVID-19, subject to fulfilling other inclusion and exclusion criteria, were randomized to receive either SOC or test drug Artemisinin 500 mg capsule/day for 5 days+standard of care (SOC) per cycle with the option to repeat as needed until symptoms of the disease are resolved, up to a total of 3 cycles (“5 days treatment, 5 days off” comprise a cycle) or standard of care (SOC).

The subjects were randomly assigned and received treatment with either the test plus SOC (n=80) or the SOC (n=41). After having obtained signed, written Informed Consent, these subjects had undergone a screening examination. Provided all inclusion/exclusion criteria were fulfilled, the subjects were enrolled and randomized by block randomization to one of the two treatment arms on Day 1. All the subjects had received the usual treatment according to ICMR (Indian Council of Medical Research) and other Indian ministry of healthcare guidelines.

Assessments of safety and efficacy variables were performed as per the study protocol. The final visit end of study (EOS) was on Day 28. Serious AEs were followed-up until they resolve or get stabilized or until 30 days from the subject's involvement in the study had ended, whichever occurred first, and it was documented according to ICH-GCP and Indian GCP guidelines.

Over all 122 patients were screened. The subjects were randomly assigned to one of the two groups: Artemisinin 500 mg (BD) with SOC or SOC in a ratio of 2:1. Subjects were initially hospitalized for 5 days of study treatment in both treatment arms. When necessary, subjects were permitted to remain for a few more additional days in the hospital for the second cycle or third cycle of treatment.

Objective: To develop TGF-β therapeutics (TGF-beta antisense/OT-101; and TGF-β inhibitor/Artemisinin) against long term symptoms of COVID-19. Description of effort: 1) Artemisinin (ART) completed ARTI-19 trial and can be currently deployed in India as herbal supplement for respiratory health and data are being collected using a post marketing survey (PMS) platform with a single source of truth, as AI-cough analysis. OT-101 has completed a phase 2 trial in Latin America against mild to severe COVID-19 with data analysis to support its adaptive Phase 2/3 global registration trial.

Benefits of Proposed Technology: Currently treatments for COVID-19 do not consider long term symptoms arising from organ scarring and fibrosis due to organ damages and over production of TGF-β during active COVID-19. TGF-β inhibitor as adjunct to SOC would fill that gap. Oral ART for mild/moderate COVID-19 and intravenous OT-101 for severe COVID-19. By targeting the host protein-we are unlikely to generate mutational resistance against the drug. And it can be applicable to other viruses utilizing the same pathways such as other zoonoses in the future.

Artemisinin (PULMOHEAL) is a sesquiterpene lactone obtained from Artemisia annua. It is small molecule inhibitor of TGF-β. Dosing=5 day on/5 day off up to three cycles of 500 mg gelatin capsule.

OT-101 (Trabedersen) OT-101 is an antisense against TGF-β2 and inhibits TGF-β2 directly and TGF-β1 indirectly. It has completed six clinical trials in oncology with good clinical efficacy data and demonstrated suppression of IL-6. COVID-19 pts are similar to cancer pts in their pathology and as expected, OT-101 did not show any unexpected adverse events in COVID-19 pts.

What is claimed is:

1. A process for treating or ameliorating the symptoms of age disease in a human subject or animal in need, the process comprising:
preparing a pharmaceutical composition comprising an agent for inhibiting or suppressing expression of TGF-β; and
administering a therapeutically sufficient amount of the composition to the subject.

2. Use of a composition comprising an agent for inhibiting or suppressing expression of TGF-β for treating or ameliorating the symptoms of an age disease in a human subject or animal.

3. Use of a composition comprising an agent for inhibiting or suppressing expression of TGF-β in the preparation of a medicament for treating or ameliorating the symptoms of an age disease in a human subject or animal.

4. The process or use of any of claims 1-3, wherein the age disease is due to a viral disease.

5. The process or use of any of claims 1-3, wherein the age disease is due to SARS, MERS, Coronavirus, HIV, Ebola, Cytomegalovirus, human herpes virus type 6, herpes simplex virus HSV-1, herpes simplex virus HSV-2, Epstein-Ban virus, Hepatitis B virus, Enterovirus D68, or Influenza A.

6. The process or use of any of claims 1-3, wherein the age disease is long term coronavirus disease, hyperinflammatory immune disease, severe respiratory disease due to viral infection, age-related fibrotic disease, or age-related cancer.

7. The process or use of any of claims 1-3, wherein the subject is hospitalized according to one of the following:
WHO COVID-19 Clinical Improvement Ordinal Scale Criteria 3, wherein the subject is hospitalized without oxygen therapy;
WHO COVID-19 Clinical Improvement Ordinal Scale Criteria 4, wherein the subject is hospitalized with oxygen by mask or nasal prongs;
WHO COVID-19 Clinical Improvement Ordinal Scale Criteria 5, wherein the subject is hospitalized with non-invasive mechanical ventilation or high-flow oxygen; and
WHO COVID-19 Clinical Improvement Ordinal Scale Criteria 6, wherein the subject is hospitalized with intubation and mechanical ventilation.

8. The process or use of any of claims 1-3, wherein the subject has age greater than 60 years and is hospitalized and presenting at least one medical risk factor selected from:
absolute lymphocyte count≤1000 cells/mm³;
hypertension;
diabetes;
cardiac failure; and
COPD.

9. The process or use of any of claims 1-3, wherein the subject has age greater than 35 years and is hospitalized and exhibiting low PaO₂ less than 77 mmHg.

10. The process or use of any of claims 1-3, wherein the disease is multiorgan fibrosis due to aging including any one of lung failure, cardiac failure, kidney failure, and brain cognitive dysfunction.

11. The process or use of any of claims 1-3, wherein the subject has long term COVID disease symptoms due to any COVID variant.

12. The process or use of any of claims 1-3, wherein the administration or use of the composition is combined with a standard of care treatment for the disease.

13. The process or use of any of claims 1-3, comprising any one or more additional medicaments comprising anti-inflammatories, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

14. The process or use of any of claims 1-3, wherein the subject upon administration or use has an improved clinical score based on an eight point WHO COVID-19 Clinical Improvement Ordinal Scale at Day 14.

15. The process or use of any of claims 1-3, wherein the subject upon the administration or use has an improved inflammatory biomarker.

16. The process or use of any of claims 1-3, wherein the administration or use of the composition decreases mortality rate at Day 7, or Day 14, or Day 28.

17. The process or use of any of claims 1-3, wherein the administration or use of the composition improves viral load knockdown at Day 7.

18. The process or use of any of claims 1-3, wherein the administration or use of the composition increases survival rate at Day 14, or Day 28.

19. The process or use of any of claims 1-3, wherein the agent is an antisense oligonucleotide or inhibitor specific for TGF-β1, TGF-β2, or TGF-β3.

20. The process or use of any of claims 1-3, wherein the agent for inhibiting or suppressing expression of TGF-β is selected from TGF-β2-specific antisense oligonucleotides SEQ ID NOs:1-9 as follows:
SEQ ID NO: 1
gtaggtaaaa acctaatat,
SEQ ID NO: 2
gttcgtttag agaacagatc,
SEQ ID NO: 3
taaagttcgt ttagagaaca g,
SEQ ID NO: 4
agccctgtat acgac,
SEQ ID NO: 5
gtaggtaaaa acctaatat,
SEQ ID NO: 6
cgtttagaga acagatctac,
SEQ ID NO: 7
cattgtagat gtcaaaagcc,
SEQ ID NO: 8
ctccctcatg gtggcagttg a,
SEQ ID NO: 9
cggcatgtct attttgta,
and chemically-modified variants thereof, artemisinin extract, a pharmaceutically-acceptable salt, salt polymorph, ester, or isomer thereof, an artemisinin formulation, and any combination thereof.

21. The process or use of any of claims 1-3, wherein the agent for inhibiting or suppressing expression of TGF-β is an artemisinin formulation, comprising 90-95% pure artemisinin extract, or a pharmaceutically-acceptable salt, salt polymorph, ester, or isomer thereof, and one or more pharmaceutically acceptable excipients.

22. The process or use of claim 21, wherein the excipients comprise any one or more pharmaceutically acceptable excipients selected from diluents, stabilizers, disintegrants and anticaking agents.

23. The process or use of claim 21, wherein the excipients comprise any one or more of microcrystalline cellulose, polysorbate 80, crospovidone, croscarmellose sodium, and magnesium stearate.

24. The process or use of any of claims 1-3, wherein the agent for inhibiting or suppressing expression of TGF-β is an artemisinin compound or derivative thereof, or a pharmaceutically-acceptable salt, salt polymorph, ester, or isomer thereof.

25. The process or use of any of claims 1-3, wherein the agent for inhibiting or suppressing expression of TGF-β is a compound, or ligand comprising a small molecule or polypeptide, that interacts with Site I of TGF-β comprising Trp30 and/or Site II of TGF-β comprising Arg15, G1n19, and Phe8, or a pharmaceutically-acceptable salt, salt polymorph, ester, or isomer thereof.

26. The process or use of any of claims 1-3, wherein the agent for inhibiting or suppressing expression of TGF-β is a polypeptide or peptide mimetic of Site I of TGF-β comprising residues Phe24-Lys37 and/or Site II of TGF-β comprising residues Cys7-G1n19, or a pharmaceutically-acceptable salt, salt polymorph, ester, or isomer thereof.

27. The process or use of any of claims 1-3, wherein the agent for inhibiting or suppressing expression of TGF-β is an antibody or antibody fragment with affinity for Site I of TGF-β comprising residues Phe24-Lys37 and/or Site II of TGF-β comprising residues Cys7-G1n19.

28. The process or use of any of claims 1-3, wherein the agent for inhibiting or suppressing expression of TGF-β is a compound comprising a sesquiterprene lactone or derivative thereof, or a pharmaceutically-acceptable salt, salt polymorph, ester, or isomer thereof.

29. The process or use of any of claims 1-3, wherein the agent for inhibiting or suppressing expression of TGF-β is a compound comprising three isoprenyl groups and one lactone ring, or derivative thereof, or a pharmaceutically-acceptable salt, salt polymorph, ester, or isomer thereof.

30. The process or use of any of claims 1-3, wherein the composition is prepared from a lyophilized powder of the agent.

31. The process or use of any of claims 1-3, wherein the agent is a TGF-β2-specific antisense oligonucleotide selected from SEQ ID NOs:1-9 and chemically-modified variants thereof, and administered or used by continuous intravenous infusion at a dose of 140 mg/m 2 on Days 1 to 7.

32. The process or use of any of claims 1-3, wherein the agent is a TGF-β2-specific antisense oligonucleotide selected from SEQ ID NOs:1-9 and chemically-modified variants thereof, and administered or used by continuous intravenous infusion with a Cmax value of 2-3 μg/mL.

33. The process or use of any of claims 1-3, wherein the agent is a TGF-β2-specific antisense oligonucleotide selected from SEQ ID NOs:1-9 and chemically-modified variants thereof, and administered or used by continuous intravenous infusion at a dose of 140 mg/m 2 on Days 1 to 7, either singly or in combination with artemisinin in any form at a dose of 500 mg per day on Days 1 to 5.

34. The process or use of any of claims 1-3, comprising suppressing symptoms due to TGF-β induced proteins upon administration or use of the composition.

35. The process or use of any of claims 1-3, comprising suppressing symptoms due to any one of long term coronavirus disease, hyperinflammatory immune disease, severe respiratory disease due to viral infection, age-related fibrotic disease, or age-related cancer upon administration or use of the composition.

36. The process or use of any of claims 1-3, comprising suppressing symptoms due to multiorgan inflammatory syndrome, cytokine storm, vasculitis, or Kawasaki syndrome upon administration or use of the composition.

37. The process or use of any of claims 1-3, comprising suppressing symptoms due to cytokine storm upon administration or use of the composition.

38. The process or use of any of claims 1-3, comprising reducing intensive care unit duration upon administration or use of the composition.

39. The process or use of any of claims 1-3, comprising reducing hospitalization duration upon administration or use of the composition.

40. The process or use of any of claims 1-3, comprising increasing ventilator-free days upon administration or use of the composition.

41. A pharmaceutical composition for inhibiting or suppressing expression of TGF-β, or for inhibiting or suppressing entry or replication of a virus in a cell, or for inhibiting or suppressing an inflammatory response or cytokine storm, or for treating or ameliorating the symptoms of an age-related disease in a human subject or animal, the composition comprising:
a TGF-β inhibitor, artemisinin, pharmaceutically acceptable salts forms, esters, polymorphs or stereoisomers thereof, and any combination thereof; and
a carrier.

42. The composition of claim 41, wherein the TGF-β inhibitor is selected from TGF-β2-specific antisense oligonucleotides SEQ ID NOs:1-9 and chemically-modified variants thereof.

43. The composition of any of claims 41-42, wherein the carrier is sterile water for injection, saline, isotonic saline, or a combination thereof.

44. The composition of any of claims 41-42, wherein the composition is substantially free of excipients.

45. The composition of any of claims 41-42, wherein the composition is stable for at least 14 days in carrier at 37° C.

46. The composition of any of claims 41-42, wherein the composition is combined with a standard of care medicament for the disease.

47. The composition of any of claims 41-42, comprising any one or more additional medicaments comprising anti-inflammatories, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

48. The composition of any of claims 41-42, wherein the age disease is due to a viral disease.

49. The composition of any of claims 41-42, wherein the age disease is due to SARS, MERS, Coronavirus, HIV, Ebola, Cytomegalovirus, human herpes virus type 6, herpes simplex virus HSV-1, herpes simplex virus HSV-2, Epstein-Ban virus, Hepatitis B virus, Enterovirus D68, or Influenza A.

50. The composition of any of claims 41-42, wherein the age disease is long term coronavirus disease, hyperinflammatory immune disease, severe respiratory disease due to viral infection, age-related fibrotic disease, or age-related cancer.

51. The composition of any of claims 41-42, wherein the disease is multiorgan fibrosis due to aging including lung failure, cardiac failure, kidney failure, or brain cognitive dysfunction.

52. A kit comprising a lyophilized powder in a vial at a content of 250 mg each of one or more TGF-β2-specific antisense oligonucleotides selected from SEQ ID NOs:1-9 and chemically-modified variants thereof.

53. A kit comprising a lyophilized powder in a vial at a content of 500 mg of artemisinin or a derivative thereof, or a compound, or ligand comprising a small molecule or polypeptide, that interacts with Site II of TGF-β comprising Arg15, Gln19, and Phe8, a sesquiterprene lactone or derivative thereof, or a compound comprising three isoprenyl groups and one lactone ring and derivatives thereof, or a pharmaceutically-acceptable salt, salt polymorph, ester, or isomer thereof, or any combination of any of the foregoing.
